(19) United States
(12) Patent Application Publication
(10) Pub. No.: US 2022/0047408 A1
(43) Pub. Date: Feb. 17, 2022

(54) CHEST STRAPPING THERAPY VEST

(71) Applicant: RespiVest LLC, Denver, CO (US)

(72) Inventors: Sai K. Medi, Centennial, CO (US); Farhood Firouzbakht, Denver, CO (US)

(21) Appl. No.: 17/399,261

(22) Filed: Aug. 11, 2021

Related U.S. Application Data
(60) Provisional application No. 63/064,966, filed on Aug. 13, 2020.

(51) Int. Cl. A61F 5/03

(52) U.S. Cl. CPC A61F 5/03

(57) ABSTRACT

A chest strapping therapy vest is disclosed. The chest strapping therapy vest includes two or more chest straps configured to each maintain an independently adjustable tension level. Each chest strap includes a series of teeth on a first end and a ratchet buckle on a second end. The ratchet buckle is configured to receive the first end of the chest strap and releasably engage the series of teeth. The chest strapping therapy vest also includes two shoulder straps, each configured to receive and adjustably position the two or more chest straps.

### CLAIM OF PRIORITY UNDER 35 U.S.C. § 119

The present application for patent claims priority to Provisional Application No. 63,064,966 entitled “CHEST STRAPPING THERAPY VEST” filed Aug. 13, 2020 and assigned to the assignee hereof and hereby expressly incorporated by reference herein.

### BACKGROUND

### Field

The present disclosed embodiments relate generally to respiratory disease therapy, and more specifically to devices and methods for chest wall strapping respiratory disease therapy.

### Background

Patients with bronchospastic and air trapping diseases, such as chronic obstructive pulmonary disease (COPD), emphysema, asthma, or reactive airway disease (RAD), typically experience difficulty expelling air from their lungs. Traditionally, the primary focus of therapy for such patients has been to open up airways to increase expiratory flow; however, more recently, Chest Strapping Therapy (CST) has also been shown to be an effective expiration aid.

In CST, straps are wrapped tightly around the patient's chest applying pressure on the chest wall to reduce chest compliance, lung compliance, and lung volume during respiration while increasing lung elastic recoil. As a result, CST allows a patient to expel air more efficiently from the lungs during expiration.

However, there is currently no apparatus that enables patients to undergo CST at home and in a clinical setting, and there is no commonly accepted or FDA approved method of therapy that exists utilizing CST.

There is therefore a need in the art for a new CST apparatus and method of therapy that enable patients to receive CST in home, clinical, and hospital settings while allowing for a great deal of adjustability so that the CST may be optimized and tailored for each patient's sizing and respiratory needs.

### SUMMARY

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

Some aspects of the present disclosure may be characterized as a chest strapping therapy vest with two or more chest straps configured to each maintain an independently adjustable tension level. Each chest strap includes a series of teeth on a first end and a ratchet buckle on a second end. The ratchet buckle is configured to receive the first end of the chest strap and releasably engage the series of teeth, temporarily locking the chest strap at a specific tension level. The chest strapping therapy vest further includes two shoulder straps, and each shoulder strap is configured to receive and adjustably position the two or more chest straps.

Other aspects of the present disclosure may be characterized as a chest strapping therapy vest with two or more chest straps configured to each maintain an independently adjustable tension level. Each chest strap includes means for releasably engaging and locking together a first end and a second end of the chest strap and means for displaying a tension level of the chest strap associated with the locking together of the first end and the second end. The chest strapping therapy vest further includes two shoulder straps, and each shoulder strap is configured to receive and adjustably position the two or more chest straps.

Other aspects of the present disclosure may be characterized as a method for applying a chest strapping therapy vest. The method includes providing a chest strapping therapy vest that includes two or more chest straps configured to each maintain an independently adjustable tension level. Each chest strap includes means for releasably engaging and locking together a first end and a second end of the chest strap and means for displaying a tension level of the chest strap associated with the locking together of the first end and the second end. The chest strapping therapy vest further includes two shoulder straps, and each shoulder strap includes a plurality of sleeves positioned at various heights with height position markings indicative of a height position of each of the sleeves. One or more of the plurality of sleeves associated with a first height position are configured to receive a first chest strap of the two or more chest straps, and one or more of the plurality of sleeves associated with a second height position are configured to receive a second chest strap of the two or more chest straps. The method further includes placing each of the two shoulder straps over a corresponding shoulder of a user and inserting each of the two or more chest straps into corresponding sleeves of the two shoulder straps associated with a particular height position and a particular height position marking, encircling the user. The method further includes tightening the two or more chest straps to initial tension levels using the means for releasably engaging and locking together and tightening, using the means for releasably engaging and locking together, the two or more chest straps to match specific, optimized tension levels using the means for displaying to determine current tension levels of the two or more chest straps.

### BRIEF DESCRIPTION OF THE DRAWINGS

FIGS. 1A and 1B illustrate, respectively, a front and back view of a CST vest in accordance with one or more embodiments described herein;

FIG. 2 illustrates strap positioning of a CST vest, in accordance with one or more embodiments described herein;

FIG. 3 illustrates an exemplary buckle-lever strap locking mechanism of a CST vest, in accordance with one or more embodiments described herein;

FIGS. 4A, 4B, and 4C illustrate an exemplary embodiment of a CST vest configured for low-profile wear, in accordance with one or more embodiments described herein, wherein FIGS. 4A and 4B illustrate, respectively, a front and back view of the CST vest in a closed state, and FIG. 4C illustrates a front view of the CST vest in an open state;

FIG. 5 illustrates an exemplary ratchet buckle strap locking mechanism of a CST vest, in accordance with one or more embodiments described herein;

FIG. 6 illustrates an exemplary method for applying a CST vest, in accordance with one or more embodiments described herein;

FIG. 7 illustrates an exemplary method for applying a CST vest configured for low-profile wear, in accordance with one or more embodiments described herein; and

FIG. 8 is an exemplary block diagram depicting physical components that may be used to implement “intelligent” aspects described herein.

### DETAILED DESCRIPTION

The word “exemplary” is used herein to mean “serving as an example, instance, or illustration.” Any embodiment described herein as “exemplary” is not necessarily to be construed as preferred or advantageous over other embodiments.

The present disclosure may enable users to improve their respiratory functionality through an adjustable CST vest that can be applied effectively in both a home and clinical setting. Additionally, the present disclosure may enable both users and medical professionals to configure the CST vest to achieve a specific, optimized tension level in chest straps through a titration process utilizing respiratory biometrics. Furthermore, the present disclosure may enable users to repeatably achieve the specific, optimized tension level in the chest straps in future applications of the CST vest. By implementing the apparatuses and methods of the present disclosure, significant improvements in CST may be obtained, potentially improving patient outcomes and quality of life.

In some embodiments, a CST vest is configured to apply pressure to the chest wall of a user. The CST vest may have an upper-chest portion above the armpits and a lower-chest portion extending below the armpits to the 7^{th} or 8^{th} intercostal space of the user. The upper-chest portion may comprise two adjustable shoulder straps configured to maintain and display a specified shoulder strap tension level. For example, each shoulder strap may comprise two opposing patches of hook and loop fastening material (e.g., VELCRO brand) configured to overlap and maintain tension in the shoulder strap based on a degree of overlap of the hook and loop fastening material and display a shoulder strap tension level via markings, such as numerical markings, configured to correlate a degree of overlap of the hook and loop fastening material with shoulder strap tension level, the markings being adjacent to one of the hook and loop fastening material patches. The lower-chest portion may be coupled to two or more chest straps configured to maintain and display an adjustable, specified chest strap tension level while encircling the user. Each chest strap may comprise a buckle-lever locking mechanism on one end and a series of teeth on an opposing end. The teeth may be configured to receive a buckle of the buckle-lever locking mechanism, enabling for tension to be placed on the chest strap. A lever mechanism of the buckle-lever locking mechanism may be configured to place further tension on the chest strap after the buckle has been received by the teeth and lock the chest strap in the resulting heightened state of tension. Such a buckle-lever locking mechanism may provide a means for releasably engaging and locking together a first end and a second end of the chest strap. Markings adjacent to the teeth, such as numerical markings, may be configured to display chest strap tension level based on the location of the buckle within the teeth after the buckle-lever locking mechanism has been locked. Such markings may provide a means for displaying a tension level of the chest strap associated with the locking together of the first end and the second end.

The adjustability and markings associated with the shoulder straps and chest straps may enable users to repeatably achieve a specific tension level in the straps across multiple applications of the CST vest in a variety of settings. For example, a user or medical professional may determine a specific, optimized tension level in both the shoulder straps and chest straps through a calibrating titration process utilizing one or more respiratory biometrics, which may include the results of pulmonary function tests (PFTs), such as peak flow readings and forced expiratory volume, inspiratory/expiratory ratio (I/E ratio), respiratory rate, severity of wheezing, and heartrate. I/E ratio may be a particularly useful biometric as expiration is often prolonged in bronchospastic and air trapping diseases causing a reduced I/E ratio, which the CST vest may correct if calibrated optimally to aid in expiration. The shoulder strap and chest strap tension levels may be marginally altered while measuring the resulting change in respiratory functionality using the one or more respiratory biometrics to eventually determine a specific, optimized tension level for each strap that does not cause the user excessive discomfort. The specific, optimized tension level of each strap may then be repeatedly achieved in future applications of the CST vest.

The CST vest may potentially be used in both short-term and long-term bronchospastic and air trapping disease therapy. For example, several preset CST vests of varying sizes may be stored in an emergency room to be used in the treatment of acute bronchospastic events. In such an emergency acute bronchospastic event, one of the preset CST vests may be selected based on the body dimensions of the user, and a coarse-calibrating titration of the shoulder and chest strap tension levels may be conducted based on at least one of subjective patient improvement, severity of wheezing, and respiratory rate. In more long-term therapy applications, the CST vest may be custom fitted and undergo a more precise calibrating titration of the shoulder and chest strap tension levels using more specific respiratory biometrics. Additionally, long-term users may potentially have devices for measuring respiratory biometrics at home to inform and enable for minor optimizing adjustments in tension levels outside of a clinical setting.

In some embodiments, the CST vest may comprise a respiratory rate sensor. For example, the respiratory rate sensor may be realized by two wireless sensors integrated into the main body of the CST vest and configured to sense a change in distance between the two wireless sensors. The first sensor may be positioned mid-sternum near the 4^{th} or 5^{th} intercostal space, and the second sensor may be positioned below the left armpit overlying the 6^{th} or 7^{th} rib. Both sensors may be configured to contact the skin of the user without overlapping with the chest straps of the CST vest. In another example, the two wireless sensors may be integrated directly into the chest straps of the CST vest. The two wireless sensors may communicate with each other via, for example, Bluetooth or ANT+, and may sense the chest wall movement of the user during respiration as a change in the distance between the two wireless sensors. The duration of user inspiration and expiration may be determined using the measured change in distance and be used to calculate the I/E ratio. Both of the wireless sensors may communicate information relating to the respiratory rate and I/E ratio to a computing device, such as a smartphone, which may process and distribute the information to aid in assessing the effectiveness of the current CST vest calibration settings. For example, a medical professional may be alerted remotely if the respiratory rate or I/E ratio reaches an unsafe level. It is also contemplated that the CST vest may be augmented with a pulse oximeter to enable local and/or remote monitoring (e.g., IoT-type remote monitoring) of pulse oximetry. The pulse oximeter may be integrated within the CST vest or added as an attachable accessory.

In some embodiments, the CST vest may additionally comprise a heart rate monitor, which may be integrated into the respiratory rate sensor wireless communication system potentially providing additional useful information regarding the respiratory function of the user and the effectiveness of the current CST vest calibration settings. For example, an abnormally high heart rate may suggest that the user is getting inadequate ventilation and the CST vest should be recalibrated.

In some embodiments, the CST vest may comprise a pressure transducer, which may be integrated into the respiratory rate sensor wireless communication system. For example, the pressure transducer may be integrated into a chest strap of the CST vest and positioned against the anterior chest wall of a user. The pressure transducer may communicate information, such as information relating to pressure applied by the CST vest to the chest wall of a user, to a computing device, such as a smartphone. Such information may potentially aid in the CST vest calibration process and in ensuring that the CST vest maintains specific, optimized tension levels after calibration.

In other embodiments, the present disclosure may comprise a CST vest configured to apply pressure to the chest wall of a user and configured for low-profile wear, such as under clothing. The CST vest may comprise two or more chest straps each configured to maintain an independently adjustable tension level. Each chest strap may comprise a series of teeth on a first end and a ratchet buckle on a second end. The ratchet buckle may be configured to receive the first end of the chest strap and releasably engage the series of teeth, temporarily locking the chest strap at a specific tension level. Such a ratchet buckle locking mechanism may provide a means for releasably engaging and locking together the first end and the second end of the chest strap. For example, the chest strap may encircle a user and maintain a specific, adjustable tension level while a releasably engageable body of the ratchet buckle is engaged with the teeth, which may have a saw-tooth configuration, locking the ends of the chest strap together. The releasably engageable body may be configured to releasably engage the series of teeth upon slidable insertion of the first end into the ratchet buckle, such as through a biasing mechanism. The ratchet buckle may be disengaged from the teeth by pressing a button on the ratchet buckle allowing the ends of the chest strap to be separated. The button may be configured to disengage the releasably engageable body from the series of teeth when depressed, such as by counteracting the bias of the biasing mechanism. In another example, each chest strap may comprise tension level markings adjacent to the series of teeth indicative of a tension level associated with a particular location along the series of teeth when the particular location is releasably engaged with the ratchet buckle. Such tension level markings may provide a means for displaying a tension level of the chest strap associated with the locking together of the first end and the second end.

The CST vest may also comprise two shoulder straps each configured to receive and adjustably position the two or more chest straps, such as in a “suspender” configuration with the two shoulder straps draped over a user's shoulders and extending downward along the user's chest. For example, each shoulder strap may comprise a plurality of sleeves positioned at various heights and configured to receive the two or more chest straps. Each shoulder strap may also comprise height position markings on the plurality of sleeves indicative of a height position of each sleeve. For example, one or more of the plurality of sleeves associated with a specific height position (i.e., on the same horizontal plane) may be configured to receive one of the two or more chest straps. As in the previously described embodiments, a first chest strap of the two or more chest straps may be configured to be positioned around the 5^{th} intercostal space of a user, and a second chest strap of the two or more chest straps may be configured to be positioned around the 7^{th} intercostal space of the user. Furthermore, the CST vest may comprise at least one of a heart rate monitor, a respiratory rate sensor, and a pressure transducer, which may be integrated into the two or more chest straps, for example, potentially aiding in CST vest calibration and the monitoring of CST vest efficacy.

As with other embodiments, the adjustability and markings associated with the shoulder straps and chest straps may enable users to repeatably achieve a specific tension level and position in the chest straps across multiple applications of the CST vest. Additionally, a specific, optimized tension level in the two or more chest straps may be determined through a calibrating titration process utilizing one or more respiratory biometrics as previously described. Furthermore, various features of the disclosed embodiments may be used interchangeably, such as the buckle-lever locking mechanism and the ratchet buckle locking mechanism.

In some embodiments, the CST vest may comprise two or more chest straps configured to each maintain an independently adjustable tension level. Each chest strap may comprise a means for releasably engaging and locking together a first end and a second end of the chest strap, such as a series of teeth on the first end and a ratchet buckle on the second end configured to receive the first end of the chest strap and releasably engage the series of teeth, temporarily locking the chest strap at a specific tension level. For example, a releasably engageable body of the ratchet buckle may engage the teeth, which may have a saw-tooth configuration, locking the ends of the chest strap together. The releasably engageable body may be configured to releasably engage the series of teeth upon slidable insertion of the first end into the ratchet buckle, such as through a biasing mechanism. The ratchet buckle may be disengaged from the teeth by pressing a button on the ratchet buckle allowing the ends of the chest strap to be separated. The button may be configured to disengage the releasably engageable body from the series of teeth when depressed, such as by counteracting the bias of the biasing mechanism. Additionally, each chest strap may comprise a means for displaying a tension level of the chest strap associated with the locking together of the first end and the second end, such as tension level markings adjacent to the means for releasably engaging and locking together indicative of the tension level associated with a particular location of the locking together of the first end and the second end.

The CST vest may also comprise two shoulder straps, each shoulder strap configured to receive and adjustably position the two or more chest straps. For example, each shoulder strap may comprise a plurality of sleeves positioned at various heights and configured to receive the two or more chest straps. Each shoulder strap may also comprise height position markings on the plurality of sleeves indicative of a height position of each sleeve, and one or more of the plurality of sleeves associated with a specific height position may be configured to receive one of the two or more chest straps. For example, a first chest strap of the two or more chest straps may be configured to be positioned around the 5^{th} intercostal space of a user, such as by being received by one or more of the plurality of sleeves associated with a first specific height position, and a second chest strap of the two or more chest straps may be configured to be positioned around the 7^{th} intercostal space of the user, such as by being received by one or more of the plurality of sleeves associated with a second specific height position. The CST may also comprise at least one of a heart rate monitor, a respiratory rate sensor, and a pressure transducer, which may be integrated into the two or more chest straps, for example, potentially aiding in CST vest calibration and the monitoring of CST vest efficacy.

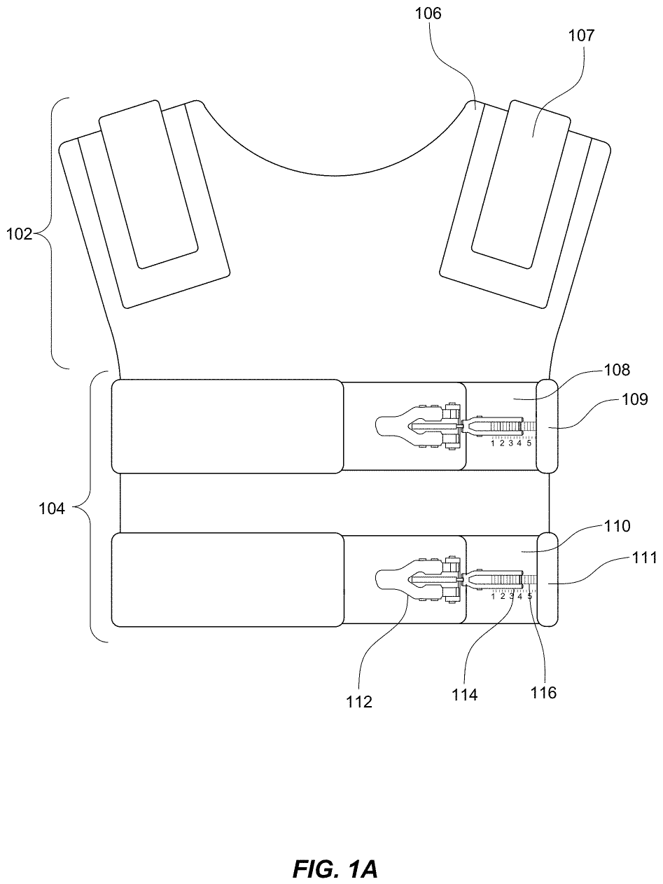

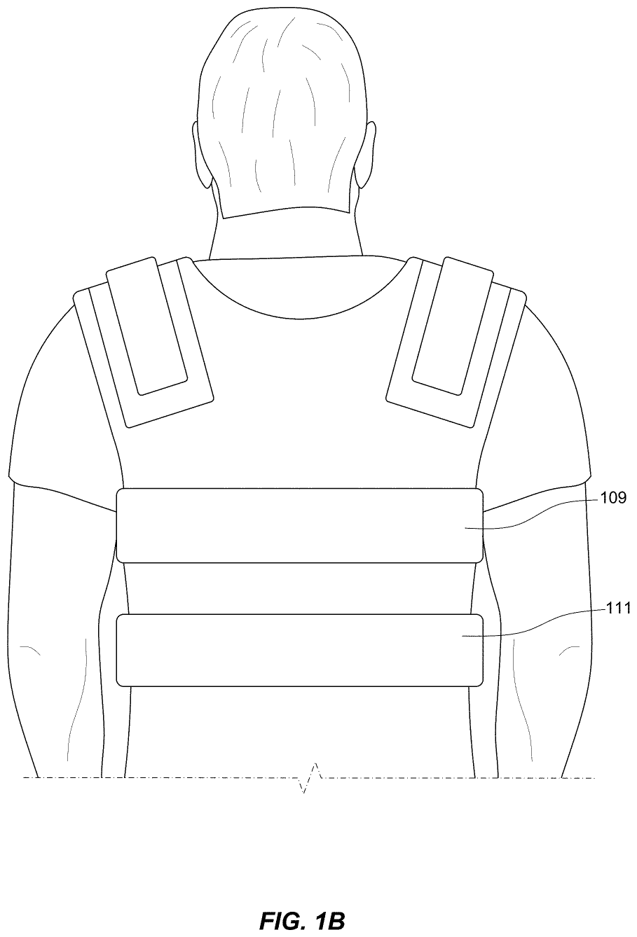

Referring now to the drawings, FIGS. 1A and 1B illustrate, respectively, a front and back view of an exemplary embodiment of a CST vest. The CST vest may have an upper-chest portion 102 above the armpits and a lower-chest portion 104 extending below the armpits to the 7^{th} or 8^{th} intercostal space of the user. The upper-chest portion 102 may be constructed of one or more semi-rigid materials, such as a fabric composite or semi-rigid foam, with the back segment being more rigid than the front segment. The relatively more rigid back segment of the upper-chest portion 102 may enable the CST vest to aid users in maintaining posture by keeping the clavicles in place, which may improve respiration, particularly in users who hunch their backs as is often seen in severe asthma cases for example. The upper-chest portion 102 may comprise two adjustable shoulder straps 106 configured to maintain and display an adjustable, specified shoulder strap tension level. Each shoulder strap 106 may comprise two opposing hook and loop (e.g., VELCRO brand) patches 107 configured to overlap and maintain tension in the shoulder strap 106 based on degree of patch 107 overlap and display a shoulder strap 106 tension level via markings configured to correlate degree of patch 107 overlap with shoulder strap 106 tension level, the markings being adjacent to the patches 107. The markings may include tick marks with numerical labels indicative of the shoulder strap 106 tension level. The shoulder straps 106 may enable the CST vest to limit outward expansion of the scapulae and clavicles during respiration, which may aid in respiration efficiency, particularly in users with bronchospastic or air trapping diseases, and the markings may enable the shoulder straps 106 of the CST vest to repeatably achieve an adjustable, specified tension level.

The lower-chest portion 104 may be constructed of one or more elastic materials and may include two or more horizontal sleeves, or seams, such as upper horizontal sleeve 109 and lower horizontal sleeve 111, configured to receive an upper chest strap 108 and a lower chest strap 110, respectively. The upper chest strap 108 and lower chest strap 110 may be configured to maintain and display an adjustable, specified tension level while encircling the user. The upper chest strap 108 and lower chest strap 110 may be constructed of a relatively wide, such as a 2-3 inch width, non-elastic fabric with overlapping ends and may have padding on the side facing the chest wall of the user, enabling for an increased comfort level for the user. The upper chest strap 108 and lower chest strap 110 each may comprise a buckle-lever locking mechanism, which may have both a lever mechanism 112 and a buckle 114, on one end and a series of teeth 116 on the opposing end. The teeth 116 may be configured to receive the buckle 114 of the buckle-lever locking mechanism, enabling for tension to be placed on the upper chest strap 108 and lower chest strap 110. The lever mechanism 112 of the buckle-lever locking mechanism may be configured to place further tension on the upper chest strap 108 and lower chest strap 110 after the buckle 114 has been received by the teeth 116 and to lock the upper chest strap 108 and lower chest strap 110 in the resulting heightened state of tension, enabling users to limit the potential of dislodging the buckle 114. Markings, such as tick marks with numerical labels indicative of tension level, adjacent to the teeth 116 may be configured to display the upper chest strap 108 and lower chest strap 110 tension level based on the location of the buckle 114 within the teeth 116 after the buckle-lever locking mechanism has been locked. The adjustability and markings associated with the shoulder straps 106, upper chest strap 108, and lower chest strap 110 may enable users to repeatably achieve a specific tension level for each strap across multiple applications of the CST vest in a variety of settings.

In other embodiments, a third chest strap may be received by a third horizontal sleeve of the lower-chest portion 104 between the upper chest strap 108 and the lower chest strap 110. The third chest strap may comprise all the features of the upper chest strap 108 and lower chest strap 110 and may enable a more optimal strap configuration to increase respiratory efficiency, particularly in users with large or long bodily habitus or when additional pressure on the chest wall is required. The necessity of the third strap may be determined by a medical professional.

In some embodiments, the CST vest may be sized according to the bodily habitus and sex of the user. User chest dimension measurements may be taken and used to construct a CST vest fitted for a particular user. Such custom sizing of the CST vest may enable for an increased operational efficiency and comfort level for the user, particularly in users wearing the CST vest for long periods of time.

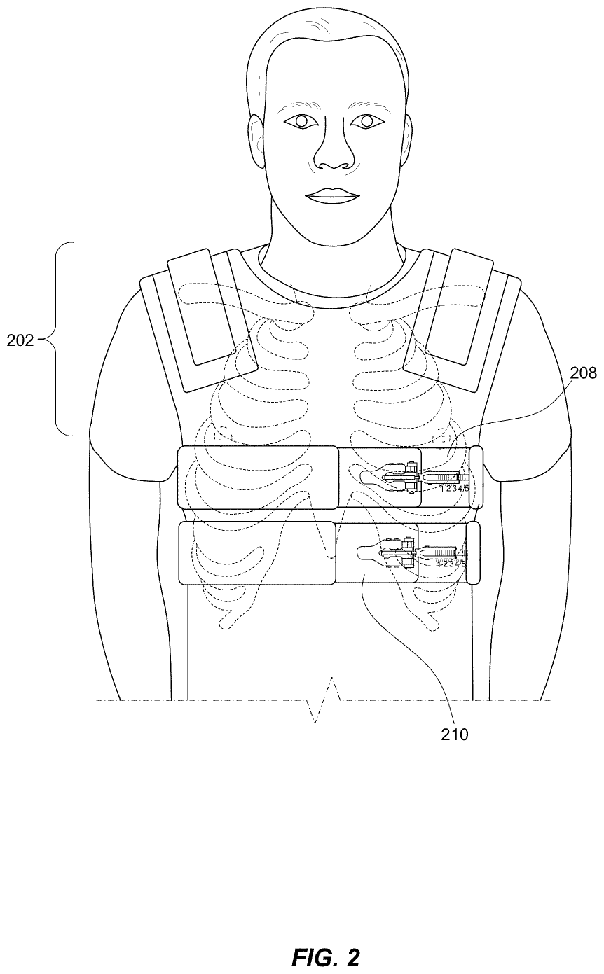

FIG. 2 illustrates the strap positioning of an exemplary embodiment of a CST vest, such as the CST vest of FIGS. 1A and 1B. The CST vest may have an upper-chest portion 202 with adjustable shoulder straps situated in the upper-chest region that extends from the armpits upward and over the clavicles. The CST vest may also have an upper chest strap 208 situated below the armpits in the mid-chest region that surrounds the 5^{th} intercostal space. The upper chest strap 208 may be placed against the lower pectoralis muscles just underneath the nipples. The CST vest may also have a lower chest strap 210 situated in the lower-chest region that surrounds the 7^{th} intercostal space. The lower chest strap 210 may be placed directly over the terminal portion of the sternum.

In other embodiments, the positioning, number, and dimensions of the chest straps used may vary depending on the bodily habitus and sex of the user. The removability of the chest straps from the CST vest may enable for a wide variety of chest straps to be interchanged quickly and easily according to the needs of the user. A medical professional may determine the optimal strap characteristics to be used for each user.

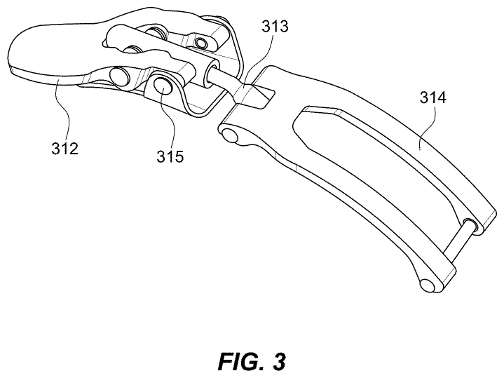

FIG. 3 illustrates a buckle-lever strap locking mechanism of an exemplary embodiment of a CST vest, such as the CST vest of FIGS. 1A and 1B. On each chest strap, the CST vest may have a buckle-lever locking mechanism, which may have both a lever mechanism 312 and a buckle 314, on one end and a series of teeth on an opposing end. The teeth may be configured to receive the buckle 314 with the buckle 314 being seated in a recess between adjacent teeth, enabling for tension to be placed on the chest straps. The buckle 314 may comprise an arcuate section with a hinge portion configured to couple with the lever mechanism via a pin 313. The lever mechanism of the buckle-lever locking mechanism may be configured to place further tension on the chest straps by rotating around a hinge 315 of the lever mechanism and pulling on the pin 313 after the buckle 314 has been received by the teeth and to lock the chest straps in the resulting heightened state of tension, enabling users to limit the potential of dislodging the buckle 314. The buckle-lever locking mechanism may operate in a similar fashion to, for example, the buckle-lever mechanism seen in ski boots.

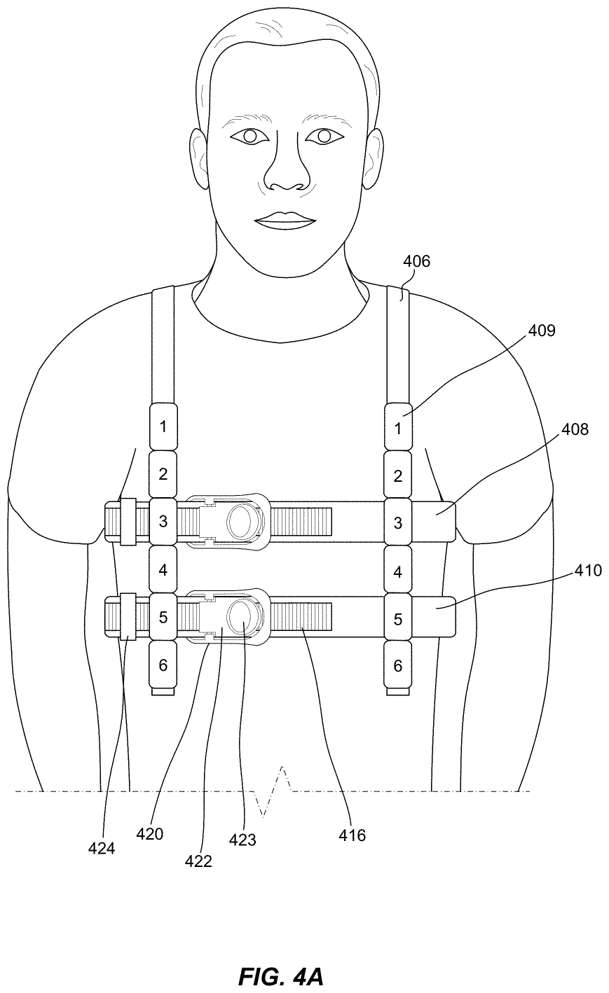

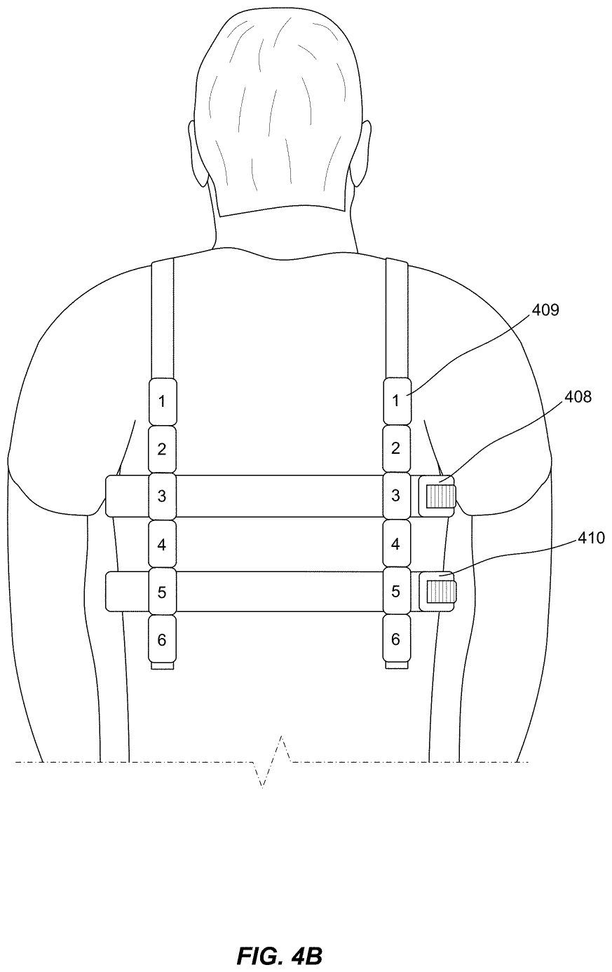

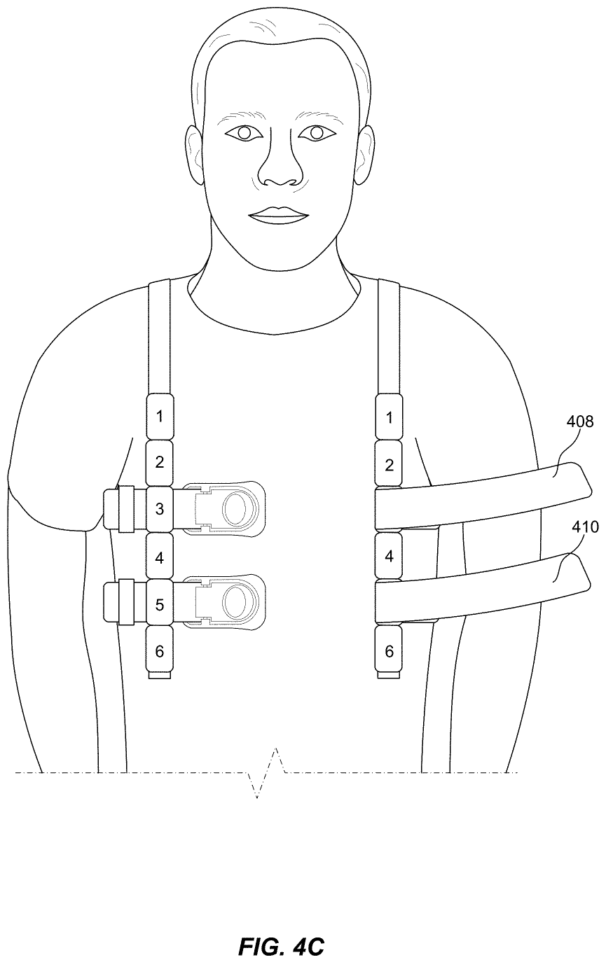

FIGS. 4A-4C illustrate an exemplary embodiment of a CST vest configured for low-profile wear, such as under clothing. The CST vest may comprise an upper chest strap 408 and a lower chest strap 410, each configured to maintain and display an independently adjustable, specified tension level while encircling a user. The upper chest strap 408 and lower chest strap 410 may be constructed of a relatively wide, such as a 2-3 inch width, non-elastic fabric with overlapping ends and may have padding on the side facing the chest wall of the user, which may be encapsulated in a soft fabric, such as Nylon, enabling for an increased comfort level for the user. The upper chest strap 408 and the lower chest strap 410 may each comprise a series of teeth 416 on a first end and a ratchet buckle 420 on a second end. For example, the series of teeth 416 and ratchet buckle 420 may be constructed substantively of a plastic material and coupled to each chest strap via strong adhesives or by sewing the material of each chest strap around components of the series of teeth 416 and ratchet buckle 420, such as to form an enclosing loop. The ratchet buckle 420 may be configured to slidably receive the first end of the corresponding chest strap and releasably engage the series of teeth 416, temporarily locking the chest strap at a specific tension level. For example, a releasably engageable body 422 of the ratchet buckle 420 may be biased by a biasing mechanism, such as a spring or other elastic material, of the ratchet buckle 420 to releasably engage the series of teeth 416 upon slidable insertion of the first end of the chest strap. The series of teeth 416 may have a saw-tooth configuration that engages with the releasably engageable body 422 preventing the chest strap from sliding out of the ratchet buckle 420 and locking the ends of the chest strap together, while still allowing the chest strap to slide further through the ratchet buckle 420. The ratchet buckle 420 may have a button 423 configured to disengage the releasably engageable body 422 from the series of teeth 416 when depressed allowing the ends of the chest strap to be separated. For example, the button 423 may couple to a lever mechanism of the ratchet buckle 420 that counteracts the bias of the biasing mechanism when the button 423 is depressed raising and disengaging the releasably engageable body 422 from the series of teeth 416. Such a ratchet buckle locking mechanism may provide a means for releasably engaging and locking together the first end and the second end of each chest strap.

Additionally, the upper chest strap 408 and the lower chest strap 410 may each comprise an adjustable clip 424 configured to hold a section of each chest strap that may protrude from the ratchet buckle 420 upon engagement with the series of teeth 416. For example, the clip 424 may be constructed of plastic and may slidably encircle the second end of the corresponding chest strap as well as the protruding section that overlaps the second end. Furthermore, the upper chest strap 408 and the lower chest strap 410 may each comprise tension level markings, such as numerical labels, adjacent to the series of teeth 416 indicative of a tension level associated with a particular location along the series of teeth 416 when the particular location is releasably engaged with the ratchet buckle 420. Such tension level markings may provide a means for displaying a tension level of each chest strap associated with the locking together of the first end and the second end. The adjustability and tension level markings associated with the upper chest strap 408 and the lower chest strap 410 may enable users to repeatably achieve a specific tension level in the chest straps across multiple applications of the CST vest. Additionally, a specific, optimized tension level in the upper chest strap 408 and the lower chest strap 410 may be determined through a calibrating titration process utilizing one or more respiratory biometrics as previously described.

The CST vest may also comprise two shoulder straps 406 each configured to receive and adjustably position the upper chest strap 408 and the lower chest strap 410. The shoulder straps 406 may be constructed substantively of a soft fabric, such as Nylon, and may have padding along a side configured to interface with the shoulders of a user, such as under a top portion of the shoulder straps 406, enabling greater user comfort. The shoulder straps 406 may be configured to be in a “suspender” configuration with the two shoulder straps 406 draped over a user's shoulders and extending vertically downward along the user's chest. Each shoulder strap 406 may comprise a plurality of horizontal sleeves 409 positioned at various heights and configured to receive the upper chest strap 408 and the lower chest strap 410. The plurality of sleeves 409 may be labeled with height position markings, such as numerical markings, indicative of a height position of each sleeve 409. The sleeves 409 associated with a specific height position may be configured to receive either the upper chest strap 408 or the lower chest strap 410.

For example, the four sleeves 409 labeled with the height position marking “3” (two sleeves 409 shown in the FIG. 4A frontal view and two sleeves 409 shown in the FIG. 4B back view) may slidably receive the upper chest strap 408, and the four sleeves 409 labeled with the height position marking “5” may slidably receive the lower chest strap 410. The height positioning of the upper chest strap 408 may be configured to be positioned around the 5^{th} intercostal space of a user and the height positioning of the lower chest strap 410 may be configured to be positioned around the 7^{th} intercostal space of the user. The plurality of sleeves 409 with varying height positions may enable a variety of users with varying torso dimensions to adjustably position the upper chest strap 408 and the lower chest strap 410 appropriately, and the corresponding height position markings may enable repeatability of such appropriate placement. For example, a user may know their height position number and slide the upper chest strap 408 and lower chest strap 410 into sleeves 409 with a corresponding height position number. Additional chest straps or alternative chest strap positioning may be implemented for users with larger or longer torsos or for female users due to breast tissue. For example, female users may place the upper chest strap 408 in a lower position.

The configuration of the two shoulder straps 406, the upper chest strap 408, and the lower chest strap 410 enables easy access to the front chest of a user as is shown in FIG. 4C. The first and second ends of both the upper chest strap 408 and lower chest strap 410 may be easily disengaged using the button 423 of the ratchet buckle 420, and the upper chest strap 408 and lower chest strap 410 may be placed in an open configuration while still being received by the plurality of sleeves 409. Such ease of access may be of particular importance during an emergency where chest compressions or electrical cardioversion may be necessary.

Optionally, the CST vest may also comprise at least one of a heart rate monitor, a respiratory rate sensor, and a pressure transducer, each of which may be modularly integrated into the upper chest strap 408 or the lower chest strap 410 and configured to communicate information to a computing device as described above, potentially aiding in CST vest calibration and the monitoring of CST vest efficacy.

In other embodiments, the number, spacing, size, and positioning of the plurality of sleeves 409 may vary. For example, the plurality of sleeves 409 may comprise a fewer number of sleeves regularly spaced 1-2 inches apart between approximately the nipple line, or axilla, and just below the subxiphoid.

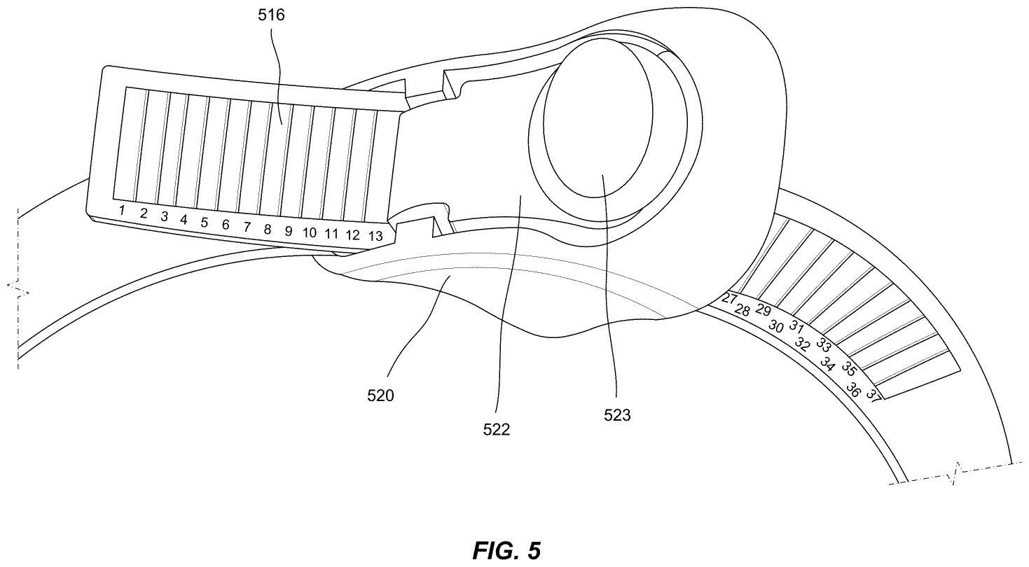

FIG. 5 illustrates an exemplary ratchet buckle strap locking mechanism of a CST vest, such as the CST vest of FIGS. 4A-4C. Each chest strap of the CST vest may have a series of saw-tooth shaped teeth 516 on a first end and a ratchet buckle strap locking mechanism, which may include a ratchet buckle 520, on a second end. The ratchet buckle 520 may be configured to slidably receive the first end of the chest strap and releasably engage the series of teeth 516, temporarily locking the chest strap at a specific tension level. A releasably engageable body 522 of the ratchet buckle 520 may be biased by a biasing mechanism of the ratchet buckle 520 to press down upon and releasably engage the series of teeth 516 upon slidable insertion of the first end of the chest strap, allowing the first end of the chest strap to slide only one direction through the ratchet buckle 520. The saw-tooth shape of the series of teeth 516 may be angled to raise the releasably engageable body 522 allowing the series of teeth 516 to pass under the releasably engageable body 522 during insertion of the first end of the chest strap into the ratchet buckle 520. The vertical face on each of the series of teeth 516 may engage the releasably engageable body 522 preventing the first end of the chest strap from sliding out of the ratchet buckle 520 and locking the ends of the chest strap together. A button 523 of the ratchet buckle 520 may couple to a lever mechanism of the ratchet buckle 520 that raises an end of the releasably engageable body 522 that is configured to engage with the series of teeth 516. When depressed, the button 523 pivots the end of the releasably engageable body 522 upward, counteracting the bias of the biasing mechanism and disengaging the releasably engageable body 522 from the series of teeth 516. Consequently, the depressing of the button 523 unlocks the ratchet buckle 520 allowing the ends of the chest strap to be separated. The chest strap may also comprise tension level markings, such as the shown numerical labels, adjacent to the series of teeth 516 indicative of a tension level associated with a particular location, such as between adjacent teeth 516, along the series of teeth 516 when the particular location is releasably engaged with the releasably engageable body 522 of the ratchet buckle 520.

In some embodiments, various features of the exemplary CST vest depicted in FIGS. 1A and 1B may be used interchangeably with features of the exemplary CST vest depicted in FIGS. 4A-4C. For example, the buckle-lever locking mechanism of FIGS. 1A and 1B may be implemented in the CST vest of FIGS. 4A-4C instead of the shown ratchet buckle locking mechanism. In another example, the ratchet buckle locking mechanism of FIGS. 4A-4C may be implemented in the CST vest of FIGS. 1A and 1B instead of the shown buckle-lever locking mechanism. In yet another example, the adjustable shoulder straps 106 of FIGS. 1A and 1B may be incorporated into the upper portions of the shoulder straps 406 of FIGS. 4A-4C. In yet another example, the shoulder straps 406 of FIGS. 4A-4C may be implemented in the CST vest of FIGS. 1A and 1B instead of the upper-chest portion 102 and incorporated into the lower-chest portion 104. In yet another example, the plurality of sleeves 409 of FIGS. 4A-4C may be implemented in the CST vest of FIGS. 1A and 1B instead of the shown upper horizontal sleeve 109 and lower horizontal sleeve 111. In yet another example, the upper horizontal sleeve 109 and lower horizontal sleeve 111 of FIGS. 1A and 1B may be implemented in the CST vest of FIGS. 4A-C instead of the plurality of sleeves 409.

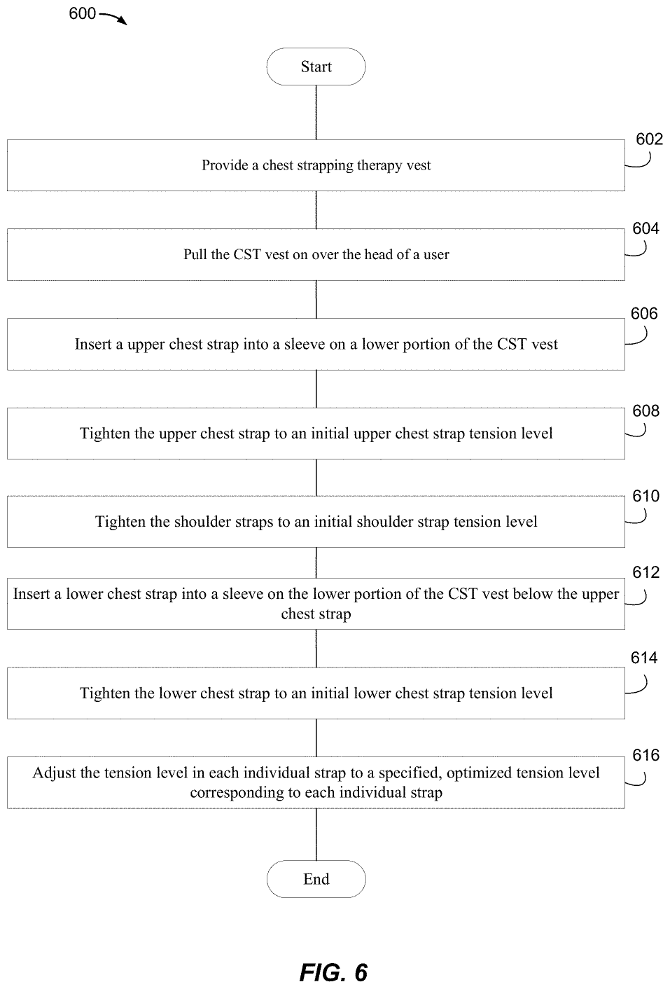

FIG. 6 illustrates an exemplary method 600 of the present disclosure for applying a CST vest, such as the CST vest of FIGS. 1A and 1B. In the context of this method, a “user” may refer to either an individual receiving the CST vest or an individual applying the CST vest. The method 600 may comprise, in block 602, providing a CST vest, such as the CST vest of FIGS. 1A and 1B. The method 600 may further comprise, in block 604, a user pulling the CST vest on over the head like a shirt and, in block 606, inserting an upper chest strap into a sleeve, or seam, on a lower portion of the CST vest. The method 600 may further comprise, in block 608, the user tightening the upper chest strap to an initial upper chest strap tension level, such as by utilizing a buckle-lever locking mechanism of the upper chest strap so that the CST vest is held in place. The method 600 may further comprise, in block 610, the user tightening shoulder straps of the CST vest to an initial shoulder strap tension level, such as by using patches of hook and loop fastener material (e.g., VELCRO brand) on the shoulder straps to lock the CST vest in place over the shoulders. The method 600 may further comprise, in block 612, the user inserting a lower chest strap into a sleeve, or seam, on the lower portion of the CST vest below the upper chest strap and, in block 614, tightening the lower chest strap to an initial lower chest strap tension level, such as by utilizing a buckle-lever locking mechanism of the lower chest strap. Blocks 602-614 may enable the CST vest to be secured to the chest of a user with the initial tension levels in the shoulder straps and chest straps. The method 600 may further comprise, in block 616, the user adjusting the tension level in each individual strap to a specified, optimized tension level corresponding to each individual strap.

In some embodiments, block 616 may further comprise determining a specific, optimized tension level for each strap through a calibrating titration process. For example, during the calibrating titration process the user may repeatedly adjust the shoulder strap and chest strap tension levels and measure the resulting change in respiratory functionality using PFT metrics, such as peak flow readings and forced expiratory volume, to eventually determine a specific, optimized tension level for each strap that does not cause the user excessive discomfort. In another example, the calibrating titration process may be a coarse calibrating titration process implemented in the case of an acute or emergency bronchospastic event in which the user may repeatedly adjust the shoulder strap and chest strap tension levels and measure the resulting change in respiratory functionality using at least one of subjective patient improvement, severity of wheezing, oxygen saturation level (obtained, e.g., via a pulse oximetry), and/or respiratory rate to eventually determine a specific, optimized tension level for each strap that does not cause the user excessive discomfort.

In some embodiments, the method 600 may further comprise the user inserting a third chest strap into a sleeve, or seam, on the lower portion of the CST vest below the upper chest strap and above the lower chest strap and tightening the third chest strap to an initial tension level utilizing a buckle-lever locking mechanism of the third chest strap. Additionally, during the calibrating titration process the user may repeatedly adjust the tension level of the third chest strap along with the other shoulder and chest straps and measure the resulting change in respiratory functionality to eventually determine a specific, optimized tension level for each strap that does not cause the user excessive discomfort.

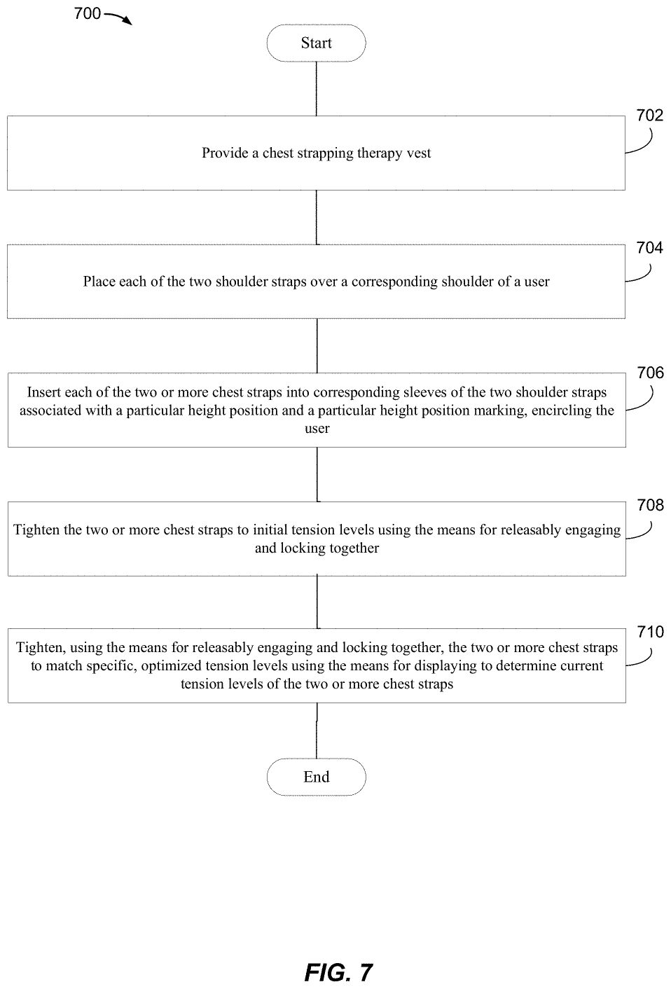

FIG. 7 illustrates an exemplary method 700 of the present disclosure for applying a CST vest configured for low-profile wear, such as the CST vest of FIGS. 4A-4C. The method 700 may comprise, in block 702, providing a CST vest, such as the CST vest of FIGS. 4A-4C. For example, the CST vest may comprise two or more chest straps configured to each maintain an independently adjustable tension level. Each chest strap may comprise a means for releasably engaging and locking together a first end and a second end of the chest strap, such as a series of teeth on the first end and a ratchet buckle on the second end configured to receive the first end of the chest strap and releasably engage the series of teeth, temporarily locking the chest strap at a specific tension level. Each chest strap may also comprise a means for displaying a tension level of the chest strap associated with the locking together of the first end and the second end, such as tension level markings adjacent to the series of teeth indicative of the tension level associated with a particular location along the series of teeth when the particular location is releasably engaged with the ratchet buckle. The CST vest may also comprise two shoulder straps, and each shoulder strap may comprise a plurality of sleeves positioned at various heights with height position markings indicative of a height position of each of the sleeves. One or more of the plurality of sleeves associated with a first height position may be configured to receive a first chest strap of the two or more chest straps, and one or more of the plurality of sleeves associated with a second height position may be configured to receive a second chest strap of the two or more chest straps.

The method 700 may further comprise, in block 704, placing each of the two shoulder straps over a corresponding shoulder of a user. For example, padded sections near the top of each of the two shoulder straps may rest on the shoulders of a user and aid in shoulder strap alignment. Additionally, the height position markings may be used to properly align the shoulder straps with the sleeves associated with a particular height position being aligned on the same horizontal plane. The method 700 may further comprise, in block 706, inserting each of the two or more chest straps into corresponding sleeves of the two shoulder straps associated with a particular height position and a particular height position marking, encircling the user. For example, the particular height position may be a specific, optimized height position, such as determined, for example, during a calibrating titration process. The method 700 may further comprise, in block 708, tightening the two or more chest straps to initial tension levels using the means for releasably engaging and locking together. For example, a first, top chest strap may be inserted into corresponding sleeves of the two shoulder straps associated with a particular height position and a particular height position marking and tightened to a top chest strap initial tension level. Then, the remaining chest straps may each be inserted into corresponding sleeves of the two shoulder straps associated with a different particular height position and a different particular height position marking for each chest strap, and each of the remaining chest straps may be tightened to a corresponding initial tension level.

The method 700 may further comprise, in block 710, tightening, using the means for releasably engaging and locking together, the two or more chest straps to match specific, optimized tension levels using the means for displaying to determine current tension levels of the two or more chest straps. For example, the specific, optimized tension levels may be known from prior calibrating titration processes, and the current tension levels of the two or more chest straps may be determined by looking at the means for displaying and altered accordingly to match the specific, optimized tension levels.

The method 700 may further comprise determining the specific, optimized tension levels and the specific, optimized height position for each of the two or more chest straps through a calibrating titration process using one or more respiratory biometrics. The one or more respiratory biometrics used may include at least one of peak flow readings, forced expiratory volume, inspiratory/expiratory ratio, respiratory rate, and heartrate. For example, during the calibrating titration process the chest strap tension levels and height positions may be repeatedly adjusted and the resulting change in respiratory functionality may be measured using respiratory biometrics, such as PFT metrics, to eventually determine the specific, optimized tension level and the specific, optimized height position for each strap that does not cause the user excessive discomfort. In another example, the calibrating titration process may be a coarse calibrating titration process implemented in the case of an acute or emergency bronchospastic event in which the chest strap tension levels and height positions may be repeatedly adjusted and the resulting change in respiratory functionality may be measured using at least one of subjective patient improvement, severity of wheezing, oxygen saturation level, and/or respiratory rate to eventually determine the specific, optimized tension level and the specific, optimized height position for each strap that does not cause the user excessive discomfort.

It is contemplated that CST vest calibration data (e.g., in terms of tension levels and/or strap settings) may be stored locally on a CST vest and/or remotely (e.g., in a user's mobile device and/or at a medical services provider's location). It is also contemplated that a history of subjective (e.g., a user-comfort-metric) and/or objective user data (e.g., respiratory rate, peak flow readings, expiratory volume, oxygen saturation levels, and/or heart rate) may be stored locally and/or remotely. The stored historical data may associate the calibration data and the user's data to analyze the user's response to the CST vest settings on an ongoing and long-term basis.

The calibrating titration and course calibrating titration processes disclosed herein may occur in a clinical or hospital setting under the direction of a medical professional utilizing pulmonary function tests (PFTs), and the calibrating titration processes may also occur in a home setting with portable PFT equipment and the guidance of a medical professional via telemedicine. As discussed further herein, many implementations of the CST vest may include one or more sensors disposed and configured to provide information indicative of a user's response (e.g., respiratory rate, oxygen saturation level, heart rate, etc.) to vest settings. The sensors may be implemented in connection with processing logic and wireless network components to realize one or more internet-of-things (IoT) devices, which are useful in the telemedicine context and may be useful in the clinical/hospital context, too.

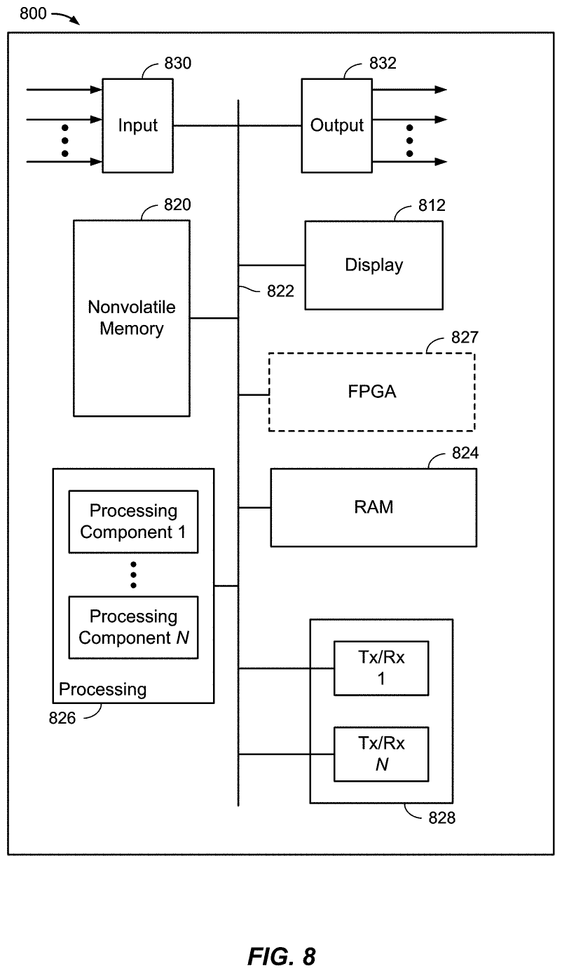

Aspects disclosed herein may be embodied directly in hardware, in processor-executable code encoded in a non-transitory tangible processor readable storage medium, or in a combination of the two. Referring to FIG. 8 for example, shown is a block diagram depicting physical components that may be utilized to realize aspects of sensors and “intelligent” logic-executing components that may be directly coupled and/or wirelessly coupled to vests disclosed herein. For example, components such as those depicted in FIG. 8 may be implemented in one or more IoT-type devices integrated within a vest. In addition, components such as those depicted in FIG. 8 may be implemented in a mobile device (e.g., a smart phone), which executes an app that communicates with remote medical service providers and sensors and/or IoT-type device(s) integrated within a vest. Thus, FIG. 8 depicts a system, which may be duplicated to realize aspects of an intelligent monitoring system. Those of ordinary skill in the art will appreciate that the constructs depicted in FIG. 8 may be realized as a system on a chip (SoC) limited in size only by fabrication techniques and/or a size of constructs required to interface with the depicted components.

As shown, in this embodiment a display 812 and nonvolatile memory 820 are coupled to a bus 822 that is also coupled to random access memory (“RAM”) 824, a processing portion (which includes N processing components) 826, an optional field programmable gate array (FPGA) 827, and a transceiver component 828 that includes N transceivers. Although the components depicted in FIG. 8 represent physical components, FIG. 8 is not intended to be a detailed hardware diagram; thus, many of the components depicted in FIG. 8 may be realized by common constructs or distributed among additional physical components. Moreover, it is contemplated that other existing and yet-to-be developed physical components and architectures may be utilized to implement the functional components described with reference to FIG. 8.

This display 812 generally operates to provide a user interface for a user, and in several implementations, the display is realized by a touchscreen display. The display 812 may also be implemented as any of a variety of simple displays such as simple LED display. In some variations of the embodiment depicted in FIG. 8 (such as a headless IoT device), the display 812 is omitted completely.

In general, the nonvolatile memory 820 is non-transitory memory that functions to store (e.g., persistently store) data and processor-executable code (including executable code that is associated with effectuating the methods described herein). In some embodiments for example, the nonvolatile memory 820 includes bootloader code, operating system code, file system code, and non-transitory processor-executable code to facilitate the execution of the methods described herein.

For example, the code may execute logic to determine: a distance between two sensors on a vest; a duration of user inspiration and expiration (using a change in the distance); and the code may be used to calculate the T/E ratio. In addition, processor-executable code may include code to utilize heart rate as a parameter value that indicative of an effectiveness of the vest's operation. It should be recognized that the logic may be implemented in the vest, in a mobile device, remotely located away from the vest and/or distributed among several local and remote devices. The nonvolatile memory 820 may also store the history of calibration data and the user's data discussed above.

In many implementations, the nonvolatile memory 820 is realized by flash memory (e.g., NAND or ONENAND memory), but it is contemplated that other memory types may also be utilized. Although it may be possible to execute the code from the nonvolatile memory 820, the executable code in the nonvolatile memory is typically loaded into RAM 824 and executed by one or more of the N processing components in the processing portion 826.

The N processing components in connection with RAM 824 generally operate to execute the instructions stored in nonvolatile memory 820 to monitoring and assessment of the vests disclosed herein. As one of ordinarily skill in the art will appreciate, the processing portion 826 may include a video processor, digital signal processor (DSP), micro-controller, graphics processing unit (GPU), or other hardware processing components or combinations of hardware and software processing components (e.g., an FPGA or an FPGA including digital logic processing portions).

Alternatively, non-transitory FPGA-configuration-instructions may be persistently stored in nonvolatile memory 820 and accessed by the processing portion 826 (e.g., during boot up) to configure the hardware-configurable portions of the processing portion 826 to effectuate the monitoring functions disclosed herein.

The input component 830 operates to receive signals (e.g., signals indicative of respiratory-rate, oxygen saturation, and heart-rate) that are indicative of a patient's status and/or a patient's response to the vest. It is also contemplated that the input component 830 may receive signals indicative of settings of the straps of the vest (e.g., to enable the settings to be transmitted to a remote medical service provider). The signals received at the input component may also include control signals from a user interface. The output component generally operates to provide one or more analog or digital signals to effectuate an operational aspect of the vests disclosed herein.

The depicted transceiver component 828 includes N transceiver chains, which may be used for communicating with external devices via wireless or wireline networks. Each of the N transceiver chains may represent a transceiver associated with a particular communication scheme (e.g., Bluetooth, ANT+, WiFi, Ethernet, etc.).

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

As used herein, the recitation of “at least one of A, B or C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

What is claimed is:

1. A chest strapping therapy vest comprising:
two or more chest straps configured to each maintain an independently adjustable tension level, each chest strap comprising:
a series of teeth on a first end; and
a ratchet buckle on a second end, the ratchet buckle configured to receive the first end of the chest strap and releasably engage the series of teeth, temporarily locking the chest strap at a specific tension level; and
two shoulder straps, each shoulder strap configured to receive and adjustably position the two or more chest straps.

2. The chest strapping therapy vest of claim 1, wherein each chest strap comprises tension level markings adjacent to the series of teeth indicative of a tension level associated with a particular location along the series of teeth when the particular location is releasably engaged with the ratchet buckle.

3. The chest strapping therapy vest of claim 1, wherein each shoulder strap comprises a plurality of sleeves positioned at various heights and configured to receive the two or more chest straps.

4. The chest strapping therapy vest of claim 3, wherein each shoulder strap comprises height position markings on the plurality of sleeves indicative of a height position of each sleeve, and one or more of the plurality of sleeves associated with a specific height position are configured to receive one of the two or more chest straps.

5. The chest strapping therapy vest of claim 1, wherein the series of teeth have a saw-tooth configuration, and the ratchet buckle comprises:
a releasably engageable body configured to releasably engage the series of teeth upon slidable insertion of the first end into the ratchet buckle; and
a button configured to disengage the releasably engageable body from the series of teeth when depressed.

6. The chest strapping therapy vest of claim 1, wherein a first chest strap of the two or more chest straps is configured to be positioned around the 5^{th} intercostal space of a user, and a second chest strap of the two or more chest straps is configured to be positioned around the 7^{th} intercostal space of the user.

7. The chest strapping therapy vest of claim 1 further comprising:
at least one of a heart rate monitor, a respiratory rate sensor, and a pressure transducer.

8. A chest strapping therapy vest comprising:
two or more chest straps configured to each maintain an independently adjustable tension level, each chest strap comprising:
means for releasably engaging and locking together a first end and a second end of the chest strap; and
means for displaying a tension level of the chest strap associated with the locking together of the first end and the second end; and
two shoulder straps, each shoulder strap configured to receive and adjustably position the two or more chest straps.

9. The chest strapping therapy vest of claim 8, wherein the means for releasably engaging and locking together includes:
a series of teeth on the first end; and
a ratchet buckle on the second end, the ratchet buckle configured to receive the first end of the chest strap and releasably engage the series of teeth, temporarily locking the chest strap at a specific tension level.

10. The chest strapping therapy vest of claim 8, wherein the means for displaying includes tension level markings adjacent to the means for releasably engaging and locking together indicative of the tension level associated with a particular location of the locking together of the first end and the second end.

11. The chest strapping therapy vest of claim 8, wherein each shoulder strap comprises a plurality of sleeves positioned at various heights and configured to receive the two or more chest straps.

12. The chest strapping therapy vest of claim 11, wherein each shoulder strap comprises height position markings on the plurality of sleeves indicative of a height position of each sleeve, and one or more of the plurality of sleeves associated with a specific height position are configured to receive one of the two or more chest straps.

13. The chest strapping therapy vest of claim 9, wherein the series of teeth have a saw-tooth configuration, and the ratchet buckle comprises:
a releasably engageable body configured to releasably engage the series of teeth upon slidable insertion of the first end into the ratchet buckle; and
a button configured to disengage the releasably engageable body from the series of teeth when depressed.

14. The chest strapping therapy vest of claim 8, wherein a first chest strap of the two or more chest straps is configured to be positioned around the 5^{th} intercostal space of a user, and a second chest strap of the two or more chest straps is configured to be positioned around the 7^{th} intercostal space of the user.

15. The chest strapping therapy vest of claim 8 further comprising:
at least one of a heart rate monitor, a respiratory rate sensor, and a pressure transducer.

16. A method for applying a chest strapping therapy vest, the method comprising:
providing a chest strapping therapy vest, the chest strapping therapy vest comprising:
two or more chest straps configured to each maintain an independently adjustable tension level, each chest strap comprising:
means for releasably engaging and locking together a first end and a second end of the chest strap; and
means for displaying a tension level of the chest strap associated with the locking together of the first end and the second end; and
two shoulder straps, each shoulder strap comprising a plurality of sleeves positioned at various heights with height position markings indicative of a height position of each of the sleeves, wherein one or more of the plurality of sleeves associated with a first height position are configured to receive a first chest strap of the two or more chest straps, and one or more of the plurality of sleeves associated with a second height position are configured to receive a second chest strap of the two or more chest straps;
placing each of the two shoulder straps over a corresponding shoulder of a user;
inserting each of the two or more chest straps into corresponding sleeves of the two shoulder straps associated with a particular height position and a particular height position marking, encircling the user;
tightening the two or more chest straps to initial tension levels using the means for releasably engaging and locking together; and
tightening, using the means for releasably engaging and locking together, the two or more chest straps to match specific, optimized tension levels using the means for displaying to determine current tension levels of the two or more chest straps.

17. The method for applying a chest strapping therapy vest of claim 16, wherein the inserting includes inserting each of the two or more chest straps into corresponding sleeves of the two shoulder straps associated with a particular height position, which is a specific, optimized height position, and a particular height position marking, encircling the user.

18. The method for applying a chest strapping therapy vest of claim 17 further comprising:
determining the specific, optimized tension levels and the specific, optimized height position for each of the two or more chest straps through a calibrating titration process using one or more respiratory biometrics.

19. The method for applying a chest strapping therapy vest of claim 18, wherein the determining includes determining the specific, optimized tension levels and the specific, optimized height position for each of the two or more chest straps through the calibrating titration process using at least one of peak flow readings, forced expiratory volume, inspiratory/expiratory ratio, respiratory rate, and heartrate.

20. The method for applying a chest strapping therapy vest of claim 16, wherein the providing includes providing the chest strapping therapy vest, the chest strapping therapy vest comprising:
the two or more chest straps configured to each maintain an independently adjustable tension level, each chest strap comprising:
means for releasably engaging and locking together the first end and the second end of the chest strap, wherein the means for releasably engaging and locking together includes:
a series of teeth on the first end; and
a ratchet buckle on the second end, the ratchet buckle configured to receive the first end of the chest strap and releasably engage the series of teeth, temporarily locking the chest strap at a specific tension level; and
means for displaying the tension level of the chest strap associated with the locking together of the first end and the second end, wherein the means for displaying includes tension level markings adjacent to the series of teeth indicative of the tension level associated with a particular location along the series of teeth when the particular location is releasably engaged with the ratchet buckle.
